(19) United States
(12) Patent Application Publication
(10) Pub. No.: US 2023/0057220 A1
(43) Pub. Date: Feb. 23, 2023

(54) ELECTROMAGNETIC NEAR FIELD RESONATOR AS SENSOR FOR ANALYTE DETECTION

(71) Applicant: SB Solutions Inc., Ulsan (KR)

(72) Inventor: Jagannath Malik, Ulsan (KR)

(73) Assignee: SB Solutions Inc., Ulsan (KR)

(21) Appl. No.: 17/535,445

(22) Filed: Nov. 24, 2021

(30) Foreign Application Priority Data
Aug. 19, 2021 (KR) 10-2021-0109290

(51) Int. Cl. A61B 5/145; A61B 5/055

(52) U.S. Cl. CPC A61B 5/14532; A61B 5/055

(57) ABSTRACT

Disclosed is an electromagnetic near field resonator as a sensor for detecting an analyte. A resonator assembly may include two feeding lines forming a resonator connected to ports and one or a plurality of H-type sensing elements formed between the two feeding lines.

### CROSS-REFERENCE TO RELATED APPLICATION

This application is based on and claims priority under 35 U.S.C. 119 to Korean Patent Application No. 10-2021-0109290, filed on Aug. 19, 2021 in the Korean intellectual property office, the disclosures of which are herein incorporated by reference in their entireties.

### TECHNICAL FIELD

The following description relates to an electromagnetic near field resonator as a sensor for detecting an analyte.

### BACKGROUND OF THE DISCLOSURE

An electromagnetic (EM)-based bio sensor for detecting a dielectric constant has already been verified to be effective in detecting a tumor and malignant tissue of a body. Dielectric characteristics of various organs and bio tissues within a living body have been previously characterized across a wide EM spectrum.

In the development of the EM-based sensor, an interest in glucose detection and measurement has risen for the past several years. Such a method may be based on the characterization of a change in the dielectric constant according to a change in the glucose concentration of blood or an interstitial fluid (ISF). In general, a change in the dielectric constant according to a change in glucose is incorporated into a change in the resonance frequency of the EM-based sensor.

An EM resonator as a bio sensor may detect a change in the dielectric constant of blood and an ISF attributable to a change in glucose. Sensor reference resonance may also vary depending on a bio environment in which the EM resonator is embedded. An EM-based sensor for blood glucose measurement was already attempted, and encouraging results have been reported in various documents.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Embodiments of the present disclosure are directed to providing a resonator assembly including one or a plurality of H-type sensing elements between two feeding lines forming a resonator connected to ports.

In an aspect, there is provided a resonator assembly including two feeding lines forming a resonator connected to ports and one or a plurality of H-type sensing elements formed between the two feeding lines.

According to an aspect, the H-type sensing element may include one or a plurality of H-type unit cells coupled with the two feeding lines.

According to another aspect, the H-type sensing element may include a metal part comprising one or a plurality of H-type slots and coupled with the two feeding lines.

According to still another aspect, the two feeding lines may include a coupling line on an identical plane supported by a flexible substrate.

According to still another aspect, the H-type sensing element may be formed on the same plane as the two feeding lines.

According to still another aspect, the resonator assembly may further include a metallic ring formed between the two feeding lines and the H-type sensing element.

According to still another aspect, coupling strength between the two feeding lines and the H-type sensing element may be adjusted by adjusting at least one of a size of the metallic ring, and a gap between the metallic ring and the two feeding lines.

According to still another aspect, if the plurality of H-type sensing elements is included, the plurality of H-type sensing elements may be periodically arranged in array as a one-dimensional structure or a two-dimensional structure.

According to still another aspect, the resonator assembly may be formed in a cylindrical form by surrounding a package including the two feeding lines and the H-type sensing element.

According to still another aspect, the resonator assembly may further include a cylindrical inner core formed to surround a package including the two feeding lines and the H-type sensing element.

According to still another aspect, a reference resonance frequency may be adjusted by adjusting at least one of a diameter or dielectric constant of the inner core.

According to still another aspect, at least one of coupling strength between the two feeding lines and the H-type sensing element and frequency tuning may be controlled by adjusting an interval between the two feeding lines and the H-type sensing element.

According to still another aspect, the resonator assembly may have a dual resonance band of less than 10 GHz.

According to still another aspect, the dual resonance band may include a first band included in a range of 2 to 4 GHz and a second band included in a range of 4 to 8 GHz.

There can be provided the resonator assembly including one or the plurality of H-type sensing elements between the two feeding lines forming the resonator connected to the port.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIGS. 1 and 2 are diagrams illustrating examples of sensors according to an embodiment of the present disclosure.

FIGS. 3 and 4 are diagrams illustrating examples of sensors each including an internal ring according to an embodiment of the present disclosure.

FIGS. 5 and 6 are diagrams illustrating other examples of sensors according to an embodiment of the present disclosure.

FIG. 7 is a diagram illustrating an example of a sensor having a cylindrical form according to an embodiment of the present disclosure.

FIG. 8 is a graph illustrating an example of a dual band resonance characteristic of a sensor according to an embodiment of the present disclosure.

FIG. 9 is a diagram illustrating an example of a sensor using one or a plurality of H-type unit cells periodically arranged as a one-dimensional structure in an embodiment of the present disclosure.

FIG. 10 is a diagram illustrating an example of a sensor using several H-type unit cells periodically arranged as a two-dimensional structure in an embodiment of the present disclosure.

FIGS. 11 and 12 are diagrams illustrating examples of sensors using one or a plurality of H type slots periodically arranged as a one-dimensional structure in an embodiment of the present disclosure.

FIG. 13 is a diagram illustrating an example of a sensor using a plurality of H type slots periodically arranged as a two-dimensional structure in an embodiment of the present disclosure.

### DETAILED DESCRIPTION

While illustrative embodiments have been illustrated and described, it will be appreciated that various changes can be made therein without departing from the spirit and scope of the disclosure.

Hereinafter, embodiments are described in detail with reference to the accompanying drawings. However, the embodiments may be changed in various ways, and the scope of right of this patent application is not limited or restricted by such embodiments. It is to be understood that all changes, equivalents and substitutions of the embodiments are included in the scope of right.

Terms used in embodiments are merely used for a description purpose and should not be interpreted as intending to restrict the present disclosure. An expression of the singular number includes an expression of the plural number unless clearly defined otherwise in the context. In this specification, it should be understood that a term, such as “include” or “have”, is intended to designate the presence of a characteristic, a number, a step, an operation, a component, a part or a combination of them described in the specification, and does not exclude the existence or possible addition of one or more other characteristics, numbers, steps, operations, components, parts, or combinations of them in advance.

All terms used herein, including technical or scientific terms, have the same meanings as those commonly understood by a person having ordinary knowledge in the art to which an embodiment pertains, unless defined otherwise in the specification. Terms, such as those commonly used and defined in dictionaries, should be construed as having the same meanings as those in the context of a related technology, and are not construed as being ideal or excessively formal unless explicitly defined otherwise in the specification.

Furthermore, in describing the present disclosure with reference to the accompanying drawings, the same component is assigned the same reference numeral regardless of its reference numeral, and a redundant description thereof is omitted. In describing an embodiment, a detailed description of a related known art will be omitted if it is deemed to make the subject matter of the embodiment unnecessarily vague.

Furthermore, in describing elements of an embodiment, terms, such as a first, a second, A, B, (a), and (b), may be used. Such terms are used only to distinguish one component from the other component, and the essence, order, or sequence of a corresponding component is not limited by the terms. When it is said that one component is “connected”, “combined”, or “coupled” to the other component, the one component may be directly connected or coupled to the other component, but it should also be understood that a third component may be “connected”, “combined”, or “coupled” between the two components.

A component included in any one embodiment and a component including a common function are described using the same name in another embodiment. Unless described otherwise, a description written in any one embodiment may be applied to another embodiment, and a detailed description in a redundant range is omitted.

Advantages of an electromagnetic (EM)-based resonator include that the resonator can be designed in various shapes and sizes, a band of the resonator can be adjusted based on various operating frequency bands, and the resonator can be optimized for an EM penetration depth and sensitivity. Measurable parameters of an EM-based sensor indicating a level of an analyte, such as glucose, may include a reflection base (S₁₁) or a transmission base (S₂₁). Both “size and phase” characteristics may be considered. The non-invasive measurement of an analyte from the outside of a body has several problems due to high reflexibility of a signal and a low penetration depth of a signal in a skin layer. This limits an interaction between a bio tissue and EM and lowers sensitivity. Furthermore, an in-body temperature is stable compared to the skin. A temperature change also affects a change in the dielectric constant. Accordingly, an implant type EM-based sensor is more stable and sensitive in detecting an analyte level.

An in-body bio sensor may also be indicated as an invasive type bio sensor, an insertion type bio sensor, or an implant type bio sensor. The in-body bio sensor may be a sensor for sensing a target analyte by using an electromagnetic wave. For example, the in-body bio sensor may measure bio information associated with a target analyte. Hereinafter, the target analyte is a material associated with a living body, and may also be indicated as a living body material or an analyte. For reference, in this specification, the target analyte is chiefly described as blood glucose, but the present disclosure is not limited thereto. The bio information is information related to a bio component of a subject, and may include a concentration, a numerical value, etc. of a target analyte, for example. If a target analyte is blood glucose, bio information may include a blood glucose numerical value.

The in-body bio sensor may measure a bio parameter (hereinafter referred to as a “parameter”) associated with the bio component, and may determine bio information from the measured parameter. In this specification, the parameter may indicate a circuit network parameter used to interpret a bio sensor and/or a bio sensing system. Hereinafter, a scattering parameter is chiefly described as an example for convenience of description, but the present disclosure is not limited thereto. For example, an admittance parameter, an impedance parameter, a hybrid parameter, a transmission parameter, etc. may be used as the parameter. In the case of the scattering parameter, a permeability coefficient and a reflection coefficient may be used. For reference, a resonance frequency calculated from the scattering parameter may be related to a concentration of a target analyte. The bio sensor may predict blood glucose by detecting a change in the permeability coefficient and/or the reflection coefficient.

The in-body bio sensor may include a resonator assembly (e.g., an antenna). Hereinafter, the resonator assembly is chiefly described as an example of the antenna. A resonance frequency of the antenna may be represented as a capacitance component and an inductance component as in Equation 1 below.

$$f = \frac{1}{2 π \sqrt{LC}} \qquad [ Equation 1 ]$$

In Equation 1, f may be a resonance frequency of the antenna included in the bio sensor using an electromagnetic wave, L may be inductance of the antenna, and C may indicate capacitance of the antenna. The capacitance of the antenna C may be proportional to a relative dielectric constant εᵣ as in Equation 2 below.

C∝εᵣ [Equation 2]

The relative dielectric constant εᵣ of the antenna may be influenced by a concentration of a surrounding target analyte. For example, if an electromagnetic wave passes through a material having a given dielectric constant, a change in amplitude and the phase may occur in the transmitted electromagnetic wave due to the reflection and scattering of the electromagnetic wave. The relative dielectric constant εᵣ may also vary because a degree of reflection and/or scattering of the electromagnetic wave is changed based on a concentration of a target analyte present around the bio sensor. This may be interpreted that bio capacitance is formed between the bio sensor and the target analyte due to a fringing field attributable to an electromagnetic wave radiated by the bio sensor including the antenna. A resonance frequency of the antenna is also changed because the relative dielectric constant. εᵣ of the antenna is changed by the concentration of the target analyte. In other words, the concentration of the target analyte may correspond to the resonance frequency.

The in-body bio sensor according to an embodiment may radiate an electromagnetic wave while sweeping a frequency, and may measure a scattering parameter according to the radiated electromagnetic wave. The in-body bio sensor may determine a resonance frequency from the measured scattering parameter, and may estimate a blood glucose numerical value corresponding to the determined resonant frequency. The in-body bio sensor may be inserted into a subcutaneous layer, and may predict blood glucose diffused from a blood vessel to an interstitial fluid.

The in-body bio sensor may estimate bio information by determining a frequency shift degree of a resonance frequency. In order to more accurately measure a resonance frequency, a quality factor may be maximized. Hereinafter, an antenna structure having an improved quality factor in an antenna device used in a bio sensor using an electromagnetic wave is described.

Three types of field areas surrounding an EM resonator (or an electromagnetic antenna) may be related to radiation energy and invalid energy. The field areas include a reactive near field area, a radiation near-field area (Fresnel area) and a remote-field area (Fraunhofer area).

A sensor (or a resonator included in the sensor) according to embodiments of the present disclosure is intrinsically non-radioactive and may have a strong near field. This can improve dielectric detection performance by minimizing energy radiation in total power supplied by the sensor. Accordingly, the sensor may operate with very low input power. The sensor may be excited in one of two ports while measuring resonance data in the other port thereof. Furthermore, the port may operate as a single port or dual port system that measures a reflection parameter. A bio environment is considered in the modeling and optimization of the sensor in a radio EM simulator.

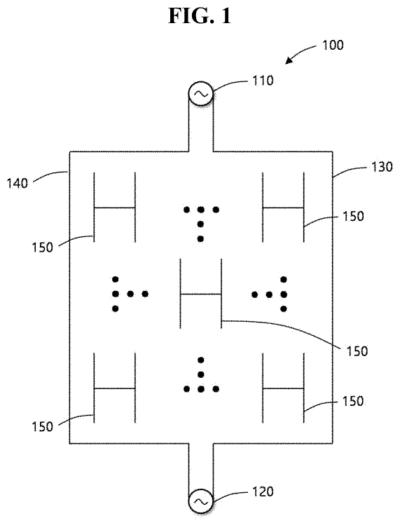

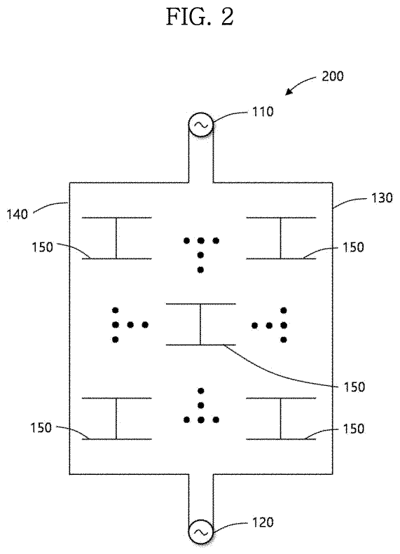

FIGS. 1 and 2 are diagrams illustrating examples of sensors according to an embodiment of the present disclosure. Each of sensors 100 and 200 of FIGS. 1 and 2 may have a periodical array of H-type unit cells 150 coupled with feeding lines 130 and 140 forming a resonator connected to two ports 110 and 120. In this case, the sensor 100 of FIG. 1 illustrates an example in which the H-type unit cells 150 are formed as a given array. The sensor 200 of FIG. 2 illustrates an example of an array in which the H-type unit cells 150 of the sensor 100 of FIG. 1 are modified. Coupling strength between the feeding lines 130 and 140 and the H-type unit cells 150 and frequency tuning can be controlled by controlling the interval between the feeding lines 130 and 140 and the H-type unit cells 150.

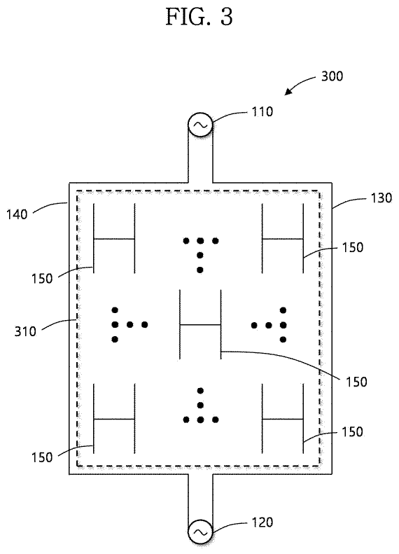

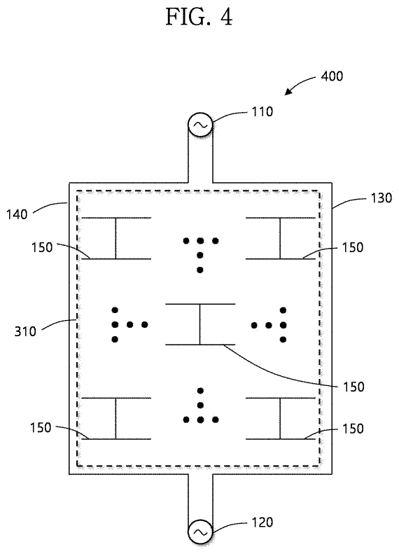

FIGS. 3 and 4 are diagrams illustrating examples of sensors each including an internal ring according to an embodiment of the present disclosure. A sensor 300 of FIG. 3 illustrates an example in which a metallic ring 310 is included in the array of the feeding lines 130 and 140 and the H-type unit cells 150 of FIG. 1. Likewise, a sensor 400 of FIG. 4 illustrates an example in which the metallic ring 310 is included in the array of the feeding lines 130 and 140 and the H-type unit cells 150 of FIG. 2. Such a metallic ring 310 may be used around the H-type unit cells 150 in order to adjust a frequency and EM field distribution on a surface of the sensor. A size of the metallic ring 310 and/or a gap between the metallic ring 310 and the feeding lines 130 and 140 may be changed in order to obtain different coupling strengths between the feeding lines 130 and 140 and the H-type unit cells 150. In other words, coupling strength between the feeding lines 130 and 140 and the H-type unit cells 150 may be adjusted by adjusting the size of the metallic ring 310.

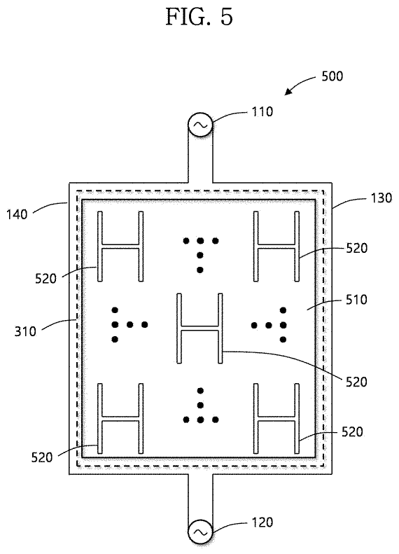

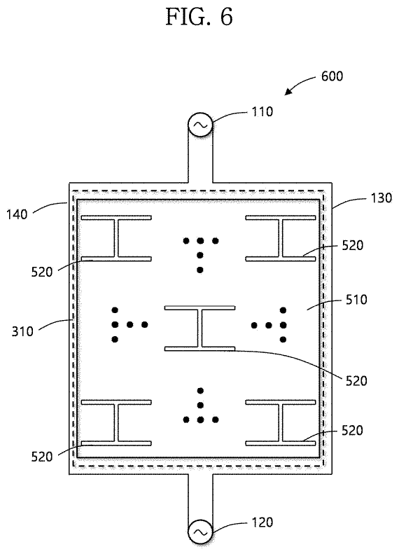

FIGS. 5 and 6 are diagrams illustrating other examples of sensors according to an embodiment of the present disclosure. Each of sensors 500 and 600 according to the present embodiment may include slot shapes 520 each formed by being cut in an H form in a metal part 510 disposed in a substrate with a slight interval an area between the two feeding lines 130 and 140. In this case, the metal part 510 may be disposed on the same surface as the feeding lines 130 and 140 on the substrate. A given interval may be formed between the metal part 510 and the feeding lines 130 and 140 so that signal ports are not disconnected. Coupling strength between the feeding lines 130 and 140 and the metal part 510 and frequency tuning may be controlled by controlling the interval. Furthermore, the metallic ring 310 may be formed in the interval.

The feeding lines 130 and 140 may include a coupling line on the same plane supported by a flexible substrate, such as thin polyamide. The feeding lines 130 and 140 may be adjusted to have proper impedance by adjusting the interval between the feeding lines 130 and 140 or changing a dielectric material of the substrate. The H-type unit cells 150 and the slot shapes 520 formed by being cut in the H type may be -type sensing elements formed on the same plane as the feeding lines 130 and 140.

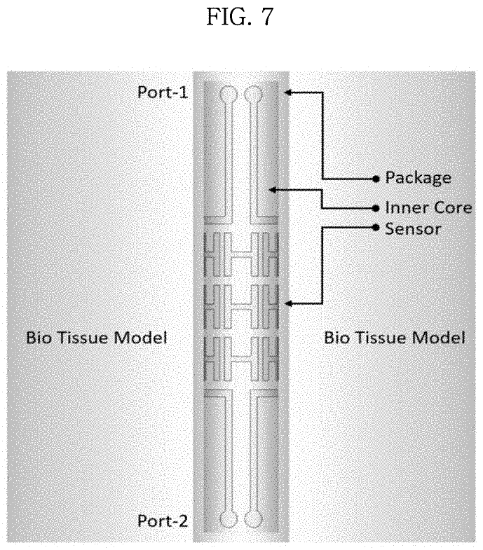

FIG. 7 is a diagram illustrating an example of a cylindrical sensor according to an embodiment of the present disclosure. A senor may be formed in a cylindrical form by surrounding the package of the sensor formed on a flexible substrate. In this case, the package may be implemented to surround a cylindrical inner core. FIG. 7 illustrates an example in which the package is implemented to surround the cylindrical inner core. The cylindrical inner core may help the sensor to maintain its shape without any change. FIG. 7 illustrates an example of a cylindrical sensor embedded in an environment of a bio tissue in order to detect an analyte from a change in the dielectric.

In this case, a reference resonance frequency of the sensor may be set by adjusting the diameter of the cylindrical inner core. Furthermore, a dielectric constant of the cylindrical inner core may be changed in order to set the reference resonance frequency of the sensor. In other words, the reference resonance frequency of the sensor may be adjusted by adjusting at least one of the diameter and dielectric constant of the cylindrical inner core.

A sensor according to embodiments of the present disclosure may chiefly have a dual resonance band of less than 10 GHz. A first resonance band may belong to an S-band (2 to 4 GHz), and a second resonance band may belong to a C-band (4 to 8 GHz). However, the two bands may be adjusted into other frequency bands by adjusting a design dimension, a substrate material, a tuning circuit and/or a matching circuit with respect to the sensor.

A sensor according to embodiments of the present disclosure may be used as a one-port or two-port construction. All scattering parameter may be measured depending on a port construction. Furthermore, both a size and a phase may be used to calculate sensitivity.

A radiation characteristic of a sensor according to embodiments of the present disclosure may have a very low strong short-distance distribution. A vibrating near field may be responsible for detecting an analyte.

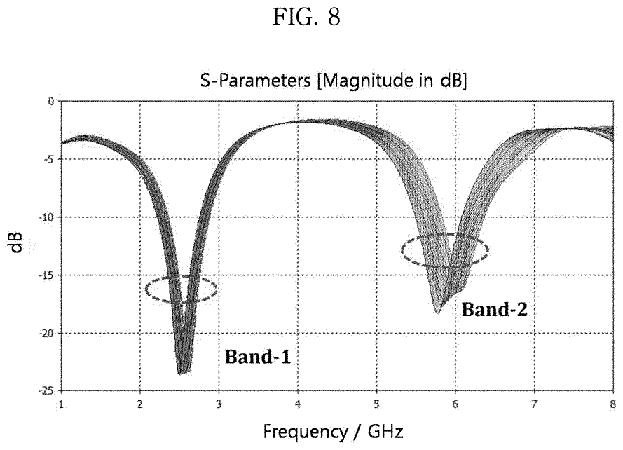

FIG. 8 is a graph illustrating an example of a dual band resonance characteristic of a sensor according to an embodiment of the present disclosure. From a graph of FIG. 8, it may be seen that two resonance bands (i.e., an S-band (i.e., a band 1 of 2 to 4 GHz) and a C-band (i.e., a band-2 of 4 to 8 GHz)) have sensitivity for a change in the dielectric constant of a dielectric.

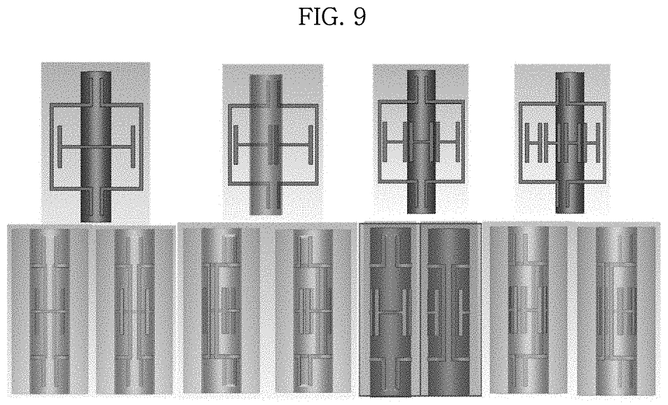

FIG. 9 is a diagram illustrating an example of a sensor using one or a plurality of H-type unit cells periodically arranged as a one-dimensional structure in an embodiment of the present disclosure. As illustrated in FIG. 9, the sensor may be implemented as a cylindrical sensor surrounding a cylindrical inner core.

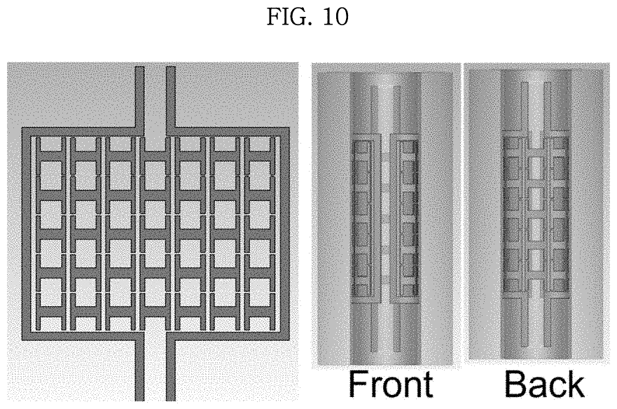

FIG. 10 is a diagram illustrating an example of a sensor using several H-type unit cells periodically arranged as a two-dimensional structure in an embodiment of the present disclosure. As illustrated in FIG. 10, the sensor may be implemented as a cylindrical sensor surrounding a cylindrical inner core.

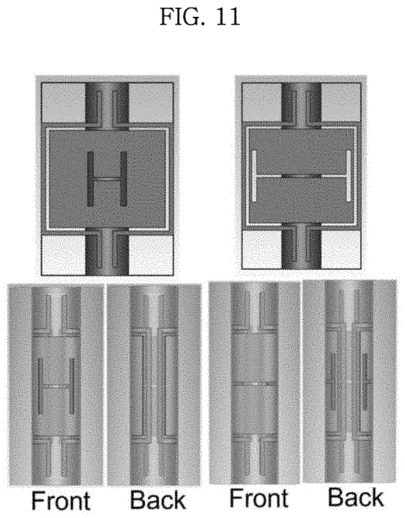

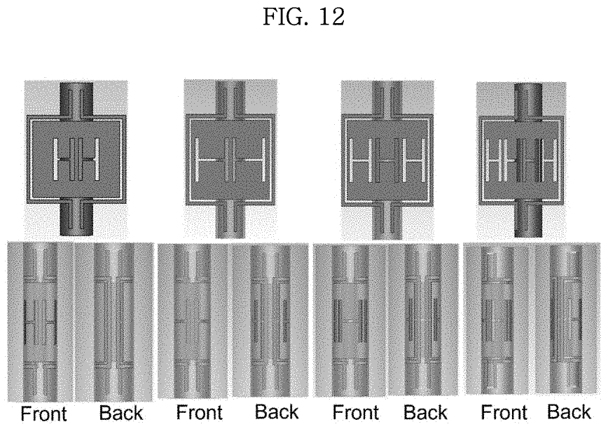

FIGS. 11 and 12 are diagrams illustrating examples of sensors each using a one or a plurality of H type slots periodically arranged as a one-dimensional structure in an embodiment of the present disclosure. As illustrated in FIGS. 11 and 12, the sensor may be implemented as a cylindrical sensor surrounding a cylindrical inner core.

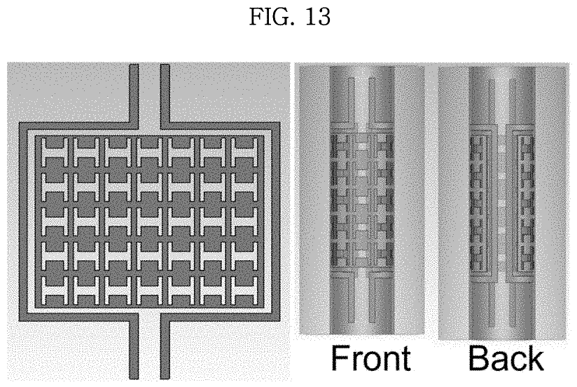

FIG. 13 is a diagram illustrating an example of a sensor using a plurality of H type slots periodically arranged as a two-dimensional structure in an embodiment of the present disclosure. As illustrated in FIG. 13, the sensor may be implemented as a cylindrical sensor surrounding a cylindrical inner core.

As described above, according to embodiments of the present disclosure, the resonator assembly including one or the plurality of H-type sensing elements between the two feeding lines forming a resonator connected to ports can be provided.

The aforementioned apparatus (or device) may be implemented as a hardware component or a combination of a hardware component and a software component. For example, the device and component described in the embodiments may be implemented using a processor, a controller, an arithmetic logic unit (ALU), a digital signal processor, a microcomputer, a field programmable gate array (FPGA), a programmable logic unit (PLU), a microprocessor or one or more general-purpose computers or special-purpose computers, such as any other device capable of executing or responding to an instruction. The processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary skill in the art may understand that the processing device may include a plurality of processing elements and/or a plurality of types of processing elements. For example, the processing device may include a plurality of processors or a single processor and a single controller. Furthermore, a different processing configuration, such as a parallel processor, is also possible.

Software may include a computer program, a code, an instruction or a combination of one or more of them and may configure a processing device so that the processing device operates as desired or may instruct the processing devices independently or collectively. The software and/or the data may be embodied in any type of machine, a component, a physical device, a computer storage medium or a device in order to be interpreted by the processor or to provide an instruction or data to the processing device. The software may be distributed to computer systems connected over a network and may be stored or executed in a distributed manner. The software and the data may be stored in one or more computer-readable recording media.

The method according to embodiments may be implemented in the form of a program instruction executable by various computer means and stored in a computer-readable medium. The computer-readable medium may include a program instruction, a data file, and a data structure solely or in combination. The medium may continue to store a program executable by a computer or may temporarily store the program for execution or download. Furthermore, the medium may be various recording means or storage means having a form in which one or a plurality of pieces of hardware has been combined. The medium is not limited to a medium directly connected to a computer system, but may be one distributed over a network. An example of the medium may be one configured to store program instructions, including magnetic media such as a hard disk, a floppy disk and a magnetic tape, optical media such as a CD-ROM and a DVD, magneto-optical media such as a floptical disk, a ROM, a RAM, and a flash memory. Furthermore, other examples of the medium may include an app store in which apps are distributed, a site in which other various pieces of software are supplied or distributed, and recording media and/or storage media managed in a server. Examples of the program instruction include a high-level language code executable by a computer by using an interpreter in addition to a machine-language code, such as that written by a compiler.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned elements, such as the system, configuration, device, and circuit, are coupled or combined in a form different from that of the described method or replaced or substituted with other elements or equivalents.

Accordingly, other implementations, other embodiments, and the equivalents of the claims fall within the scope of the claims.

1. A resonator assembly comprising:
two feeding lines forming a resonator connected to ports;
a plurality of H-type sensing elements formed between the two feeding lines, wherein the plurality of H-type sensing elements comprise a metal part comprising a plurality of H-type slots and the plurality of H-type sensing elements are coupled with the two feeding lines; and
a dual resonance band of less than 10 GHz, wherein the dual resonance band comprises a first band having a frequency ranging between 2 to 4 GHz and a second band having a frequency ranging between 4 to 8 GHz.

2. The resonator assembly of claim 1, wherein the plurality of H-type sensing elements comprise a plurality of H-type unit cells coupled with the two feeding lines.

3. (canceled)

4. The resonator assembly of claim 1, wherein the two feeding lines comprise a coupling line on an identical plane supported by a flexible substrate.

5. The resonator assembly of claim 4, wherein the plurality of H-type sensing elements is formed on a plane identical with a plane of the two feeding lines.

6. The resonator assembly of claim 1, further comprising a metallic ring formed between the two feeding lines and the plurality of H-type sensing elements.

7. The resonator assembly of claim 6, wherein coupling strength between the two feeding lines and the plurality of H-type sensing elements is adjusted by adjusting at least one of a size of the metallic ring and a gap between the metallic ring and the two feeding lines.

8. The resonator assembly of claim 1, wherein the plurality of H-type sensing elements is periodically arranged in array as a one-dimensional structure or a two-dimensional structure.

9. The resonator assembly of claim 1, wherein the resonator assembly is formed in a cylindrical form by surrounding a package comprising two feeding lines and the plurality of H-type sensing elements.

10. The resonator assembly of claim 1, further comprising a cylindrical inner core formed to surround a package comprising the two feeding lines and the plurality of H-type sensing elements.

11. The resonator assembly of claim 10, wherein a reference resonance frequency is adjusted by adjusting at least a diameter of the inner core or a dielectric constant of the inner core.

12. The resonator assembly of claim 1, wherein at least one of coupling strength between the two feeding lines and the plurality of H-type sensing elements and frequency tuning is controlled by adjusting an interval between the two feeding lines and the plurality of H-type sensing elements.

13-14. (canceled)

15. The resonator assembly of claim 1, wherein individual H-type sensing elements of the plurality of H-type sensing elements comprise two parallel sensing branches connected by a linear perpendicular sensing branch.
